(19) United States
(12) Patent Application Publication
(10) Pub. No.: US 2025/0382629 A1
(43) Pub. Date: Dec. 18, 2025

(54) RESISTANCE GENE AND LETTUCE PLANT RESISTANT TO FUSARIUM WILT

(71) Applicant: Enza Zaden Beheer B.V., Enkhuizen (NL)

(72) Inventors: Alexander Jan Teade Van Der Veen, Harderwijk (NL); Ilja Roobeek, Dirkshorn (NL); Mathieu André Pel, Utrecht (NL); Maarten Eduard Rouwet, Alkmaar (NL)

(21) Appl. No.: 18/878,271

(22) PCT Filed: Jun. 26, 2023
(86) PCT No.: PCT/EP23/67272

§ 371 Date: Dec. 23, 2024

(30) Foreign Application Priority Data
Jun. 27, 2022 (WO) PCT/EP2022/067580

(51) Int. Cl. C12N 15/82; C07K 14/415

(52) U.S. Cl. CPC C12N 15/8282; C07K 14/415

(57) ABSTRACT

The present invention relates to a lettuce plant that is resistant to Fusarium wilt, more specifically to a lettuce plant that comprises a mutated gene that confers resistance to F. oxysporum in lettuce. Furthermore the present invention relates a resistance gene and a method for obtaining a lettuce plant that is resistant to Fusarium wilt, wherein the method comprises the step of mutating a gene.

### DESCRIPTION

The present invention relates to a lettuce plant that is resistant to Fusarium wilt, more specifically to a lettuce plant that comprises a mutated gene that confers resistance to a fungus infecting lettuce. Furthermore, the present invention relates a resistance gene and a method for obtaining a lettuce plant that is resistant to Fusarium wilt, wherein the method comprises the step of mutating a gene.

Fusarium wilt is a fungal disease that is caused by Fusarium oxysporum (F. oxysporum). Fusarium wilt symptoms include wilting, chlorosis, necrosis, premature leaf drop, browning of the vascular system, stunting and damping-off, of which vascular wilt is the most important symptom showing vein clearing on the younger leaves and drooping of the older lower leaves, followed by stunting, yellowing of the lower leaves, defoliation, marginal necrosis and plant death. Fusarium wilt is a problem in many food crops affecting the production of crops worldwide including tomato, tobacco, cucurbits, sweet potatoes, banana and lettuce. In lettuce, Fusarium wilt is caused by F. oxysporum F. sp. lactucae which only affects lettuce. Although Fusarium wilt caused by F. oxysporum f. sp. lactucae affects all lettuce types it is more prevalent in crisphead, iceberg and leaf types.

F. oxysporum is a soil-borne pathogen, which can reside in the soil for long periods of time, so rotational cropping is not a useful control method. It can also spread through infected dead plant material, so cleaning up at the end of the season is important. Although the fungus appears to be sensitive to many broad-spectrum fungicides in the laboratory, these products have limited efficacy in the field. It has been proven difficult to find an effective biological control method because research in a greenhouse could not be extrapolated to the field. The best control method for F. oxysporum is the development and use of resistant varieties, although with limited success, mostly resulting in tolerance and not resistance to Fusarium wilt. A number of trials have shown that romaine types can be more tolerant to F. oxysporum infection. In contrast, crisphead, iceberg or leaf type lettuce have not demonstrated any level of resistance at all. Furthermore, although for some lettuce cultivars show tolerance to Fusarium, the pathogen under pressure will mutate to break down the disease resistance and new resistance in crops is needed to control Fusarium wilt infection.

Fusarium wilt, caused by F.oxysporum f. sp. lactucae (Fol) is the most threatening pathogen after Bremia lactucae in lettuce. Fields infested with Fol are often abandoned and lost for lettuce growth. Fol is present worldwide. At present, there are 4 different Fol races reported. Fol:1 is spread worldwide, whereas Fol:2 and Fol:3 are restricted to Japan, Taiwan and Korea. For Fol:1, Fol:2 and Fol:3, official type isolates are used by the industry to claim resistance. Fol:4 is present all-over western Europe (Portugal, Spain, Italy, France, Belgium, Netherlands, United Kingdom, Germany, Ireland and Poland). Internally (Enza Zaden, Enkhuizen, NL), furthermore two Fol:4 isolates are internally used: Fol:4_enza-AD035 (collected in NL) and Fol:4_enza-AA032 (collected in IT). Recently, the industry agreed on using the same type isolate to claim Fol:4 resistance. Fol:4_official was collected in NL and published by Gilardi et al in 2017, Plant Pathol. 66, 677-688. The Fol isolates characterized as Fol:4 seem to have the same virulence spectrum but a different level of aggressivity. Fol:4_enza-AD035 is more aggressive than Fol:4_official and Fol:4_enza-AA032.

Considering the above, there is a need in the art for to provide plants that are resistant to Fusarium wilt and wherein plants have a resistance against this pathogen. Furthermore, it is an object of present invention to provide a method to obtain such Fusarium wilt resistant plants.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a lettuce plant that is resistant to Fusarium wilt, wherein said plant comprises one or more mutations in a KINASE gene providing said resistance to Fusarium wilt, wherein said KINASE gene encodes for a KINASE protein sequence having at least 99% sequence identity, preferably 100% sequence identity with SEQ ID No. 2, wherein the plant further comprises a resistance gene LG7 that encodes for a resistance protein LG7 having at least 99% sequence identity, preferably 100% sequence identity with SEQ ID No. 8, preferably wherein said resistance protein LG7 has a valine (V) on amino acid position 111. The KINASE gene comprising the one or more mutations and providing the resistance according to the invention is hereafter referred to as KINASE R gene.

It is known that genes conferring resistance to Fusarium in tomato include gene I which encodes a leucine-rich repeat receptor-like protein (LRR-RLP), I-2 which encodes an NBS-LRR protein, I-3 which encodes an S-receptor-like kinase and I-7 which encodes a leucine-rich repeat receptor-like protein (LRR-RLP). It is expected that the KINASE R gene comprises the known NBS-LRR mechanism in the plant. The presence of the KINASE R resistance gene will decrease the chances of the pathogen overcoming the resistance, as often seen with resistance genes. Even so, combined with other resistance genes in lettuce, disease resistance (e.g. against Fusarium wilt) may even be further improved.

The identification of a novel candidate recessive resistance gene, indicated here as the KINASE R gene is obtained by gene mapping of multiple independent Fusarium wilt resistance genes in lettuce. Resistance to Fusarium was identified in a lettuce variety of L. sativa. Resistance could be mapped in the mapping population for Fusarium race 4, more specifically Fol:4_enza-AD035, on an area down to 22.922 bp in which one single recessive candidate gene, Lsa030992.1, a serine threonine protein kinase was located, referred herein as KINASE. For the first time in lettuce a KINASE gene has been found that can be linked to plant disease resistance.

According to another preferred embodiment, the present invention relates to the lettuce plant, wherein the mutations in the KINASE gene result in amino acid changes comprised of amino acid substitutions on the amino acid position 205 and/or 231 in the KINASE protein represented by SEQ ID No. 2.

According to yet another preferred embodiment, the present invention relates to the lettuce plant, wherein the mutations in the KINASE gene result in amino acid substitutions on the amino acid position 205 from a cysteine to a tyrosine C205Y, and/or on the amino acid position 231 from a tyrosine to an Asparagine Y231N.

According to another preferred embodiment, the present invention relates to the lettuce plant, wherein the KINASE gene that comprises one or more mutations encodes for the protein sequence represented by SEQ ID No. 4. The mutated KINASE gene that provided the Fusarium resistance in lettuce is referred to herein as KINASE R gene. Sequencing experiments showed that the protein encoded by the KINASE R gene from the resistant plant compared with the protein encoded by the KINASE gene of a plant that is susceptible differs in 2 amino acid substitutions, more specifically amino acid substitutions on the amino acid position 205 from a cysteine to a tyrosine C205Y, and/or on the amino acid position 231 from a tyrosine to an Asparagine Y231N. The resistance gene (KINASE R) that encodes for the protein that comprises the above mutations is represented by SEQ ID No. 3. The mutated protein is represented by SEQ ID No. 4.

SEQ ID No. 1 represents the coding sequence encoded by the KINASE gene of a susceptible Lactuca sativa. SEQ ID No. 2 represents the protein sequence encoded by the KINASE gene of a susceptible Lactuca sativa. SEQ ID No. 3 represents the coding sequence encoded by the KINASE R gene of a Fusarium wilt resistant Lactuca sativa plant of present invention. SEQ ID No. 4 represents the protein sequence encoded by the KINASE R gene of a Fusarium wilt resistant Lactuca sativa plant of present invention. SEQ ID No. 7 represents the coding sequence encoded by the resistance gene LG7 of a Fusarium wilt resistant Lactuca sativa plant of present invention. SEQ ID No. 8 represents the protein sequence encoded by the resistance gene LG7 of a Fusarium wilt resistant Lactuca sativa plant of present invention.

According to a preferred embodiment, the present invention relates to the lettuce plant, wherein the KINASE gene and resistance gene LG7 providing said resistance to Fusarium wilt are independently either heterozygous or homozygous present in the plant, preferably both the KINASE gene and the resistance gene LG7 are present homozygous. Results show that when both genes are homozygously present, the Fusarium wilt resistance in lettuce is the strongest.

According to yet another preferred embodiment, the present invention relates to the lettuce plant, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

According to another preferred embodiment, the present invention relates to the lettuce plant, wherein the lettuce plant is one or more selected from the group consisting of crisphead, butterhead, romaine, oak leaf, and Batavia. More preferably the present invention relates to the lettuce plant, wherein the lettuce plant is one or more selected from the group consisting of crisphead, butterhead, oak leaf, and Batavia. Most preferably the present invention relates to the lettuce plant, wherein the lettuce plant is a cabbage lettuce plant, wherein the cabbage lettuce plant is preferably selected from the group consisting of crisphead and butterhead lettuce. Herein, as is known to the skilled person, crisphead lettuce is equivalent to iceberg lettuce.

According to a preferred embodiment, the present invention relates to the lettuce plant, wherein the mutations in the KINASE gene are obtained by gene editing techniques, preferably by mutagenesis or CRISPR/Cas.

According to another preferred embodiment, the present invention relates to the lettuce plant, wherein Fusarium wilt is caused by Fusarium oxysporum (F. oxysporum), more preferably F. oxysporum f. sp. lactucae.

According to another preferred embodiment, the present invention relates to the lettuce plant, wherein the lettuce plant is resistant to Fusarium wilt caused by one or more of F. oxysporum selected from the group of Fol:4_official, Fol:4_enza-AD035 and Fol:4_enza-AA032,preferably at least Fol:4_official, more preferably at least Fol:4_enza-AD035 and/or Fol:4_enza-AA032. The F. oxysporum isolates have been deposited and are obtainable from deposit number NCIMB; Fol:4_Official (NCIMB 43967), Fol:4_Enza-AD035 (NCIMB 43968), and Fol:4_Enza-AA032 (NCIMB 43969). All F. oxysporum deposits have been deposited at NCIMB Ltd., Ferguson Building, Craibstone Estate, Bucksburn, Aberdeen, AB21 9YA Scotland on 30 Mar. 2022.

The present invention, according to a further aspect, relates to an F. oxysporum selected from the group of The Fol:4 enza-AD035 and/or Fol:4 enza-AA032, wherein Fol:4_Enza-AD035 is obtainable from deposit NCIMB 43968, and Fol: 4_Enza-AA032 is obtainable from deposit NCIMB 43969. The Fol:4 enza-AD035 and/or Fol:4 enza-AA032 can be used to screen and/or select for a lettuce plant that is resistant to Fusarium wilt, more specifically the lettuce plant that is resistant to Fusarium wilt according to present invention as disclosed herein.

According to a preferred embodiment, the present invention relates to the lettuce plant, wherein the resistance gene LG7 is comprised in a resistance locus LG7, wherein said resistance locus LG7 is flanked by sequences represented by SEQ ID No. 5 and SEQ ID No. 6. Experiments have shown that when KINASE R is combined with the resistance locus LG7 in lettuce comprising the resistance gene LG7, the resistance to Fusarium wilt is synergistically boosted, providing improved resistance to the more aggressive Fusarium wilt types. As with KINASE R, also the LG7 can be heterozygous or homozygous present in the plant, preferably homozygous.

According to yet another preferred embodiment, the present invention relates to the lettuce plant, wherein the mutated KINASE gene and resistance gene LG7 and resistance locus LG7 are obtainable from deposit number NCIMB 43971. Seeds comprising the mutated KINASE gene and resistance gene LG7 and resistance locus LG7 are deposited at NCIMB Ltd, Ferguson Building, Craibstone Estate, Bucksburn, Aberdeen, AB21 9YA Scotland on 6 Apr. 2022 under the number NCIMB 43971.

The present invention, according to a further aspect, relates to a seed, plant tissue, plant cell or plant part produced by or obtained from a lettuce plant of present invention. The present invention relates to a seed, plant tissue, plant cell or plant part of a lettuce plant of the present invention, comprising the KINASE gene and resistance gene and/or locus LG7 providing said resistance to Fusarium wilt as described above.

The present invention, according to a further aspect, relates to a resistance gene LG7 that confers resistance to Fusarium wilt in lettuce plants, wherein the gene comprises a coding sequence that has at least 99% sequence identity with SEQ ID No. 7 or wherein the gene encodes for a resistance protein LG7 that has at least 99% sequence identity with SEQ ID No. 8.

According to a preferred embodiment, the present invention relates to resistance gene LG7, wherein said resistance protein LG7 has a valine (V) on amino acid position 111.

The present invention, according to a further aspect, relates to a combination of a resistance gene KINASE R and a resistance locus LG7 or resistance gene LG7 as described above in a lettuce plant that confers resistance to Fusarium wilt to said lettuce plant, wherein the KINASE R gene comprises a coding sequence having at least 99% sequence identity, preferably 100% sequence identity with SEQ ID No. 3, and wherein said LG7 resistance locus comprises said resistance gene LG7, wherein said resistance gene LG7 comprises a coding sequence having at least 99% sequence identity, preferably 100% sequence identity with SEQ ID No. 7 or wherein the KINASE R gene encodes for a KINASE protein sequence having at least 99% preferably 100% sequence identity with SEQ ID No. 4, and wherein said LG7 resistance locus comprises said resistance gene LG7, wherein said resistance gene LG7 encodes for a resistance protein LG7 having at least 99%, preferably 100% sequence identity with SEQ ID No. 8.

Combining KINASE R with the resistance locus LG7 in lettuce provides improved resistance to the more aggressive Fusarium wilt types, especially Fol:4_enza-AD035 and/or Fol:4_enza-AA032.

According to another preferred embodiment, the present invention relates to the combination, wherein said KINASE R gene encodes for a KINASE R protein represented by SEQ ID No. 4 having a tyrosine (Y) on amino acid position 205 and an Asparagine (N) on amino acid position 231.

According to yet another preferred embodiment, the present invention relates to the combination, wherein said resistance gene LG7 encodes for a resistance protein LG7 having at least 99% sequence identity with SEQ ID No. 8 and wherein said resistance protein LG7 has a valine (V) on amino acid position 111.

According to a preferred embodiment, the present invention relates to the combination wherein said resistance locus LG7 is flanked by sequences represented by SEQ ID No. 5 and SEQ ID No. 6.

According to another preferred embodiment, the present invention relates to the combination, wherein the resistance to Fusarium wilt in said lettuce plant comprises resistance to F. oxysporum selected from the group of Fol:4_official, Fol:4_enza-AD035 and Fol:4_enza-AA032, preferably at least Fol:4_official, more preferably at least Fol:4_enza-AD035 and/or Fol:4_enza-AA032.

According to a preferred embodiment, the present invention relates to resistance gene KINASE R, wherein the gene encodes for a KINASE R protein that has at least 85% sequence identity with SEQ ID No. 4, preferably at least 90%, more preferably at least 95%, most preferably at least 98%, most preferably 100%.

According to another preferred embodiment, the present invention relates to the resistance gene KINASE R, wherein resistance to wilt resistance in lettuce comprises resistance to F. oxysporum selected from the group of Fol:4_official, Fol:4_enza-AD035 and Fol:4_enza-AA032, preferably at least Fol:4_official, more preferably at least Fol:4_enza-AD035 and/or Fol:4_enza-AA032.

According to yet another preferred embodiment, the present invention relates to the resistance gene KINASE R, wherein the lettuce plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

The present invention, according to a further aspect, relates to a seed produced by a lettuce plant of present invention.

The present invention, according to a further aspect, relates to a method for obtaining a lettuce plant that is resistant to Fusarium wilt, wherein the method comprises the steps of,
a) crossing a lettuce plant comprised of a combination of a resistance gene KINASE R and a resistance locus LG7 or resistance gene LG7 as disclosed herein with a lettuce plant that is not resistant to Fusarium wilt,
b) optionally, selfing the plant obtained in step a) for at least one time,
c) selecting the plants that are resistant to Fusarium wilt, more preferably resistant to F. oxysporum selected from the group of Fol:4_official, Fol:4_enza-AD035 and Fol:4_enza-AA032, preferably at least Fol:4_official, more preferably at least Fol:4_enza-AD035 and/or Fol:4_enza-AA032. In the method of the present invention the obtained lettuce plant that is resistant to Fusarium wilt is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa. The selection may involve the genotypic determination of the presence of the KINASE R gene in the plant under (a) or (b), by sequence identification of SEQ No. 4 or SEQ No. 5 in a Fusarium wilt resistant plant. The selection may also involve a phenotypic test comprises a disease resistance test against one or more of the Fol 4 isolates as indicated. Next to the determination of the KINASE R gene, the lettuce plant may be screened for the presence of the resistance locus LG7, wherein said resistance locus LG7 is flanked by sequences represented by SEQ ID No. 5 and SEQ ID No. 6. Experiments have shown that when KINASE R is combined with the resistance locus LG7 in lettuce, the resistance to Fusarium wilt is synergistically boosted, providing improved resistance to the more aggressive Fusarium wilt types. As with KINASE R, also the LG7 can be heterozygous or homozygous present in the plant, preferably homozygous.

The present invention, according to a further aspect, relates to a method for obtaining a lettuce plant that is resistant to Fusarium wilt, wherein the method comprises the step of providing one or more mutations in a KINASE gene of a lettuce plant, resulting in amino acid changes comprised of amino acid substitutions on the amino acid positions 205 and 231 in a KINASE protein having at least 99% sequence identity, preferably 100% with SEQ ID No. 2, wherein the mutations in the KINASE gene result in amino acid substitutions on the amino acid position 205 from a cysteine to a tyrosine C205Y, and on the amino acid position 231 from a tyrosine to an Asparagine Y231N, and wherein the lettuce plant further comprises an resistance gene LG7 that encodes for a resistance protein LG7 having at least 99% sequence identity, preferably 100% with SEQ ID No. 8 and wherein said resistance protein LG7 has a valine (V) on amino acid position 111. SEQ ID No.2 represents the KINASE protein sequence of a susceptible Lactuca sativa. This protein sequence does not comprise the mutations as compared to the KINASE R protein of present invention. Therefore, L. sativa that express the protein of SEQ ID No. 2 is susceptible to Fusarium wilt. The mutated KINASE protein (KINASE R) is represented by SEQ ID No. 4; comprised of amino acid substitutions on the amino acid positions 205 and/or 231 in view of the KINASE protein represented by SEQ ID No. 2.

According to another preferred embodiment, the present invention relates to the method, wherein the mutations in the KINASE gene results in a protein represented by SEQ ID No. 4.

A lettuce plant comprised of the amino acid substitutions provided a high Fusarium wilt resistance phenotype. A plant having this resistant phenotype can be obtained via use of gene editing and/or mutation techniques, such as EMS mutagenesis or CRISPR/Cas in concert with cloning techniques on the KINASE gene to generate disease resistant crops.

According to a preferred embodiment of the present invention the present plants detailed above are not plants exclusively obtained by means of an essentially biological process.

According to yet another preferred embodiment, the present invention relates to the method, wherein the mutations in the KINASE gene are obtained by gene editing techniques, preferably by mutagenesis and/or CRISPR/Cas. Alternatively, a KINASE R gene can be brought into the plant by means of transgenic techniques or by introgression.

According to another preferred embodiment, the present invention relates to the method, wherein the mutations in the KINASE gene are non-natural mutations. Mutations induced by gene editing techniques such as mutagenesis, CRISPR/Cas, transgenic techniques, or others can be regarded as non-natural mutations.

The present invention, according to a further aspect, relates to a method for identifying or selecting (i) a Fusarium wilt resistant lettuce plant or (ii) a seed of said plant as disclosed herein, wherein the method comprises the step of establishing, in the genome of said plant or seed, the presence of a resistance gene LG7 or a combination of a resistance gene KINASE R and a resistance locus LG7 or the resistance gene LG7 in a lettuce plant as defined herein.

The present invention, according to a further aspect, relates to the Use of a plasmid for introducing a combination of a KINASE R resistance gene and a resistance gene LG7 or resistance locus LG7 into the genome of a lettuce plant or lettuce plant cell, wherein the plasmid comprises the resistance KINASE R gene and the LG7 resistance gene and/or the resistance locus LG7 as disclosed herein. The KINASE R resistance gene and/or resistance locus LG7 or resistance gene LG7 of present invention may be transferred (e.g. by transformation or transfection) into plants, such as lettuce plants, using a plasmid that comprises the KINASE R resistance gene of present invention wherein the gene comprises a coding sequence that has at least 99% with SEQ ID No. 3. The resistance gene KINASE R encodes for a KINASE R protein that has at least 99% sequence identity with SEQ ID No. 4. The resistance gene KINASE R, after being transferred into the plant would provide resistance to Fusarium wilt, i.e. resistance to Fusarium Fol:4_official, Fol:4_enza-AD035 and Fol:4_enza-AA032, preferably at least Fol:4_official, more preferably at least Fol:4_enza-AD035 and/or Fol:4_enza-AA032. When KINASE R is combined with resistance gene LG7 in lettuce, the resistance to Fusarium wilt is synergistically boosted, providing improved resistance to the more aggressive Fusarium wilt types.

The present invention will be further detailed in the following examples and figures wherein:

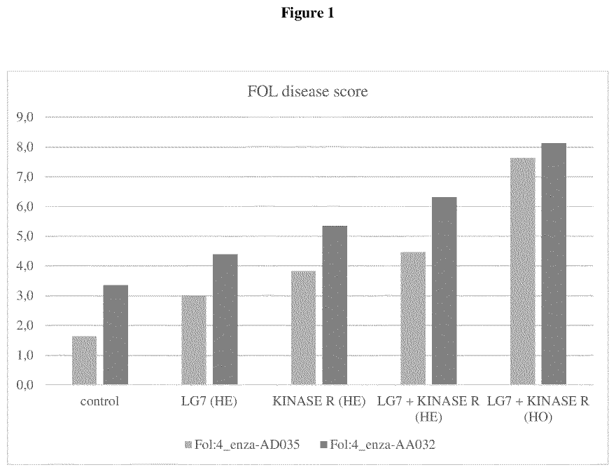

FIG. 1: shows the disease resistance score in lettuce plants comprising the KINASE R and/or LG7, either homozygous (HO) or heterozygous (HE) present in the lettuce plant. Both LG7 and the KINASE R gene provides resistance to Fol:4_enza-AD035 and Fol:4_enza-AA032. When both LG7 and KINASE R were present (homozygous or heterozygous present) in the lettuce plant, resistance to the more aggressive race Fol:4_enza-AD035 and Fol:4_enza-AA032 Fusarium wilt was further improved. When both genes are present homozygous (HO) in the lettuce plant, the highest level of Fusarium resistance was observed. A lettuce plant that does not comprise LG7 or KINASE R was used as control.

### EXAMPLES

### Gene Mapping Resistance Gene

Gene mapping experiments were done to identify a resistance gene that is involved in Fusarium (F. oxysporum f. sp. lactucae) resistance in lettuce (L. sativa). The resistance gene was originally isolated from L. sativa lettuce and was mapped on chromosome 8. The resistance gene was mapped using a Bulk Segregant Analysis (BSA) approach. The RNA of multiple F3 families that only showing Fusarium resistant plants were pooled and compared to a pool of RNA of multiple F3 families that only showed susceptible plants. SNP markers were developed and once the regions of interest could be identified and flanked by markers, a fine mapping approach was started. Markers were used in an F2 population of ˜20.000 samples to identify plants that contain a recombination between the markers, based on the specific SNPs (Table 1). On the fine-mapping on chromosome 8, the resistance area could be reduced to 22.922 bp in which one single recessive candidate gene, being a serine threonine protein kinase (KINASE R) was located (L.sat_Salinas_v9). Next to identification of the KINASE R resistance gene on chromosome 8, a further resistance locus was identified on chromosome 7, more specifically LG7.

Next to the identified KINASE R gene (SEQ ID No. 3), a resistance locus LG7 was identified that is flanked by two markers; the marker 1 (SEQ ID No. 5), having a SNP position C/T on 65092939bp and marker 2 (SEQ ID No.6), having a SNP position G/A on 67158692bp, providing a resistance locus of approximately 2M bp, which comprises several predicted resistance conferring genes. Further fine mapping resulted in a region of interest of only 50 kpb and the identification of a single resistance gene LG7 encoding for the coding sequence of SEQ ID No. 7 and resistance protein LG7 represented by SEQ ID No. 8. The resistance gene LG7 is an TIR-NBS-LRR class gene.

TABLE 1
Markers

| Marker | Sequence |
|---|---|
| Marker 1 | AGGGAAGGACTTAAGGTCCTTTAAATATGGTGC |
| (SEQ ID No. 5) | AAAACCTAAAAATTAGGGTTTTCATGTTCAGCC |
| | TCAACTCGTCGAGTAGGTTCTAGAATCCATGCG |
| | GTTTTATCAGTTTGTACACGAC |
| Marker 2 | CCAGGAAGTCAAATGAACTCCCAGGTGTGGCTC |
| (SEQ ID No. 6) | AGATAGCCAAAAAAATTTGATAGGTTCATACCC |
| | TCAAGTATGTGGGGTTTATGGTCGTAGCATAAA |
| | TTCTGCTAGACAAGGAATTCAG |

When comparing the Fusarium wilt resistant lettuce to the susceptible lettuce, base pair mutations were identified in the KINASE R gene which resulted in two amino acid changes providing the resistant phenotype in L. sativa lettuce, more specifically amino acid substitutions on the amino acid position 205 and/or 231 in the KINASE protein represented by SEQ ID No. 2. The mutations in the KINASE gene (SEQ ID No. 1) result in amino acid substitutions on the amino acid position 205 from a cysteine to a tyrosine C205Y, and/or on the amino acid position 231 from a tyrosine to an Asparagine Y231N, resulting in Fusarium resistance comprising the KINASE R gene (SEQ ID No. 3).

When comparing the Fusarium wilt resistant lettuce to the susceptible lettuce, base pair mutations were identified in the resistance gene and resistance protein LG7 (its coding sequence represented by SEQ ID No. 7, protein sequence represented by SEQ ID No. 8) which resulted in one amino acid change linked to the resistant phenotype in L. sativa lettuce, more specifically amino acid substitutions on position A111V in the LG7 protein.

### Disease Test and Biotest for Fusarium Wilt in Lettuce

Five days old cultures of F. oxysporum f. sp. lactucae, more specifically Fol:4 isolates, in Czapek Dox liquid medium were used to produce spores. The F. oxysporum isolates have been deposited and are obtainable from deposit number NCIMB; Fol:4_Official (NCIMB 43967), Fol:4_Enza-AD035 (NCIMB 43968), and Fol:4_Enza-AA032 (NCIMB 43969). After filtering the culture with cheesecloth, the spore concentration was adjusted to 5×10⁶ spores per ml. Uprooted seedlings of 10-14 days old were dipped into the spore solution and transplanted into a soil heated at 25° C. located in a glasshouse in which the air temperature is between 22° C. and 28° C. Phenotyping of the disease took place between 10-14 days after inoculation using a qualitative scoring scale (disease score):

1=plant dead
3=severe growth reduction, plant wilted and cotyledons yellow
5=moderate growth reduction, yellowing, no wilting
7=some growth reduction, plant smaller than mock inoculated control
8 to 9=no symptoms, plant size and appearance comparable to mock inoculated control

Disease resistance tests show that the KINASE R gene provides resistance to Fusarium race 4, more specifically Fol:4_enza-AD035 and Fol:4_enza-AA032 (FIG. 1). Furthermore, resistance against Fol:4_official was also tested and the results for Fol:4_official were found to be comparable to those found in respect of FOL:4_enza-AA0032. Furthermore, disease resistance test show that the LG7 also provides resistance to these Fusarium race 4. When combining LG7 and KINASE R in the lettuce plant, resistance to Fusarium wilt was further improved against the more aggressive race Fol:4_enza-AD035 and Fol:4_enza-AA032. When both genes are present homozygous (HO) in the lettuce plant, the highest level of Fusarium resistance was observed. A lettuce plant that does not comprise LG7 or KINASE R was used as positive control.

### Production of Fusarium Resistant Lettuce Plant Using Prime Editor (PE) System for Lettuce Protoplasts/Cotyledon Explants

We selected the KINASE gene that encodes for a protein sequence of SEQ ID No. 2 in lettuce to generate resistant plants of present invention comprising the C205Y and/or Y231N mutations by prime editing (PE).

PE is a new CRISPR-Cas9 based gene-editing technology used for making specific mutations in the target genome. Prime editing can introduce any specific base change required, even small defined deletions or insertions, in a broader window. PE makes use of a SpCas9H840A nickase fused to a reverse transcriptase (RT) and a 3′ elongated guide RNA (pegRNA) carrying the desired mutations to obtain the mutated resistance gene in lettuce. This versatile pegRNA is a modified sgRNA that carries a reverse transcription template and primer binding site. This pegRNA anneals to the target locus and is used by the RT as a template to introduce the desired mutations into the genome of lettuce, as was described previously for plants (Lin et al., 2020, Nature Biotechnology, Xu et al. Molecular Plant 2020 and Xu et al. Nature Plants 2022).

We made use of the PE-P2 (Prime Editor-Plant version 2) plasmid and used the sequences of the Cas9n (H840A), M-MLV RT with multiple NLS as described by Xu et al., plant codon optimized and re-synthesized commercially via Twist Bioscience. The ZmUbil promotor was replaced with the lettuce ubiquitin promotor (as described by Kawazu et al., 2019, The Horticulture Journal), to drive the Cas9H840A nickase. Compared with single guide RNAs (sgRNAs), pegRNAs have an additional 3′ extension composed of a primer binding site and a reverse-transcription template. To determine the best pegRNA sequence we made use of the web tool pegFinder as described previously (Chow et al., 2020, Nature Biomedical Engineering) (http://pegfinder.sidichenlab.org). Subsequently sequence of KINASE with and without the desired C205Y and/or Y231N mutations were selected. The top hit pegRNA was fused to the AtU6promoter and synthesized commercially via Twist Bioscience. To construct the binary vector, the PE-P2 and pegRNA cassette were cloned into the same backbone as described by Xu et al. (2020). The binary plasmid was transformed into Agrobacterium tumefaciens strain GV2260 and colonies were analyzed using PCR.

Next, lettuce cotyledon explants were transformed with the plasmid containing agrobacterium as described before (Sun et al., 2006, FEBS letters) followed by selection and regeneration. To detect targeted mutations, fragments spanning the target from genomic DNA were amplified and sequenced using the Illumina platform. Plants containing the desired mutant allele in either homozygous or heterozygous state were self-pollinated. In the following generation, plants were selected on the presence of the homozygous mutant allele and the absence of the transgene.

The mutant plants were put in a Fusarium test using a detached leaf assay and scored for disease symptoms. The expected resistant phenotype was observed and no Fusarium disease symptoms were observed in the plants comprising the mutated domains containing the desired mutant allele in either homozygous or heterozygous state. In the following generation, plants were selected on the presence of the homozygous mutant allele and the absence of the transgene.

The mutant plants were put in a Fusarium disease test and scored for disease symptoms. The expected resistant phenotype was observed and no Fusarium disease symptoms were observed in the plants comprising the mutated domains.

### Validation of Fusarium Resistant Lettuce Plant

Similar to the previous experiment above, gene editing (GE) was performed on lettuce plants comprising the Kinase R gene and the resistance locus and gene LG7. GE mutants were made in lettuce in the Kinase R gene (SEQ ID No. 3) which resulted in a premature STOP codon at position 655bp (LT16-C4), at position 690bp (LT16-A6) or at position 658 (LT16-A7).

The mutant plants were used in a Fusarium disease test and scored for disease symptoms. 24 plants per GE mutation were tested for the Fusarium wilt resistance against Fol:4_enza-AD035 with the parental lines used in the fine mapping as positive (non-mutated lettuce, LAC) and negative control (Avidius, a lettuce plant that does not comprise the resistance gene LG7 or KINASE R was used as positive control, which is known to be susceptible) on the assay. The average disease score was monitored, see Table 2.

TABLE 2
Disease tests GE mutant plants

| | Disease resistance score |
|---|---|
| Avidius | 1-2 |
| LAC | 8-9 |
| LT16-C4 | 4.5 |
| LT16-A6 | 5.5 |
| LT16-A7 | 5.5 |

The plants with the premature STOP codon in the Kinase R gene show a higher susceptibility to the race 4 than the non-mutated lettuce plant (LAC) of present invention comprising both Kinase R gene and the resistance locus and gene LG7, indicating that the Kinase R is significantly contributing to improved Fusarium wilt resistance. Also, the activity of the resistance protein LG7 on Fusarium wilt resistance in lettuce is observed, even when the Kinase R protein is not functionally present.

1. A lettuce plant that is resistant to Fusarium wilt, wherein said plant comprises one or more mutations in a KINASE gene providing said resistance to Fusarium wilt, wherein said KINASE gene encodes for a KINASE protein sequence having at least 99% sequence identity with SEQ ID NO: 2, wherein the plant further comprises a resistance gene LG7 that encodes for a resistance protein LG7 having at least 99% sequence identity with SEQ ID NO: 8.

2. The lettuce plant according to claim 1, wherein said resistance protein LG7 has a valine (V) on amino acid position 111.

3. The lettuce plant according to claim 1, wherein the one or more mutations in the KINASE gene result in amino acid changes comprising amino acid substitutions at position 205 and/or 231 in the KINASE protein set forth in SEQ ID NO: 2, and/or wherein the one or more mutations in the KINASE gene result in amino acid substitutions at position 205 from a cysteine to a tyrosine C205Y, and/or at position 231 from a tyrosine to an Asparagine Y231N.

4. (canceled)

5. The lettuce plant according to claim 1, wherein the KINASE gene that comprises the one or more mutations encodes for the protein sequence set forth in by SEQ ID NO: 4.

6. The lettuce plant according to claim 1, wherein the KINASE gene and the resistance gene LG7 providing said resistance to Fusarium wilt are independently either heterozygous or homozygous present in the plant, and/or wherein the lettuce plant is selected from the group consisting of Lactuca sativa, Lactuca virosa, Lactuca saligna, Lactuca serriola, Lactuca aculeate, Lactuca georgica, Lactuca perennis, Lactuca tatarica, and Lactuca viminea, and/or wherein the lettuce plant is one or more selected from the group consisting of crisphead, butterhead, romaine, oak leaf, and Batavia.

7. (canceled)

8. (canceled)

9. The lettuce plant according to claim 1, wherein the one or more mutations in the KINASE gene are obtained by gene editing techniques.

10. The lettuce plant according to claim 1, wherein the Fusarium wilt is caused by Fusarium oxysporum (F. oxysporum), and/or wherein the lettuce plant is resistant to Fusarium wilt caused by F. oxysporum selected from the group consisting of Fol:4_official, Fol:4 enza-AD035, and Fol:4_enza-AA032.

11. (canceled)

12. The lettuce plant according to claim 1, wherein the resistance gene LG7 is in a resistance locus LG7, wherein said resistance locus LG7 is flanked by sequences set forth in SEQ ID NO: 5 and SEQ ID NO: 6.

13. The lettuce plant according to claim 1, wherein the mutated KINASE gene, the resistance gene LG7, and the resistance locus LG7 are obtainable from deposit number NCIMB 43971.

14. A seed, plant, plant cell, or plant part produced by or obtained from the lettuce plant according to claim 1.

15. A resistance gene LG7 that confers resistance to Fusarium wilt in lettuce plants, wherein the resistance gene LG7 comprises a coding sequence that has at least 99% sequence identity with SEQ ID NO: 7, or wherein the resistance gene LG7 encodes for a resistance protein LG7 having at least 99% sequence identity with SEQ ID NO: 8, and wherein said resistance protein LG7 has a valine (V) at amino acid position 111.

16. (canceled)

17. A combination of a resistance gene KINASE R with a resistance locus LG7 or a resistance gene LG7 in a lettuce plant that confers resistance to Fusarium wilt to said lettuce plant, wherein:
the resistance gene KINASE R comprises a coding sequence having at least 99% sequence identity with SEQ ID NO: 3, and wherein said resistance locus LG7 comprises said resistance gene LG7, wherein said resistance gene LG7 comprises a coding sequence having at least 99% sequence identity with SEQ ID NO: 7; or
the resistance gene KINASE R encodes for a KINASE protein sequence having at least 99% sequence identity with SEQ ID NO: 4, and wherein said resistance locus LG7 comprises said resistance gene LG7, wherein said resistance gene LG7 encodes for a resistance protein LG7 having at least 99% sequence identity with SEQ ID NO: 8, and/or
wherein said resistance gene KINASE R encodes for a KINASE R protein set forth in SEQ ID NO: 4 having a tyrosine (Y) at position 205 and an Asparagine (N) at position 231; and/or
wherein said resistance gene LG7 encodes for the resistance protein LG7 having at least 99% sequence identity with SEQ ID NO: 8, and wherein said resistance protein LG7 has a valine (V) at position 111,
wherein the resistance to Fusarium wilt in said lettuce plant comprises resistance to F. oxysporum selected from the group consisting of Fol:4 official, Fol:4 enza-AD035, and Fol:4_enza-AA032.

18. (canceled)

19. (canceled)

20. The combination according to claim 17, wherein said resistance locus LG7 is flanked by sequences set forth in SEQ ID NO: 5 and SEQ ID NO: 6.

21. (canceled)

22. A method for obtaining a lettuce plant that is resistant to Fusarium wilt, wherein the method comprises the steps of:
(a) crossing a lettuce plant comprising the combination according to claim 17 with a lettuce plant that is not resistant to Fusarium wilt,
(b) optionally, selfing the plant obtained in step (a) for at least one time,
(c) selecting plants that are resistant to the Fusarium wilt, wherein the Fusarium wilt is caused by F. oxysporum selected from the group consisting of Fol:4_official, Fol:4_enza-AD035, and Fol:4_enza-AA032.

23. A method for obtaining the lettuce plant of claim 1, wherein the method comprises the step of;
providing the one or more mutations in a KINASE gene of a lettuce plant, resulting in amino acid changes comprising amino acid substitutions of C205Y and Y231N in the KINASE protein having at least 99% sequence identity with SEQ ID NO: 2,
wherein the mutations in the KINASE gene result in amino acid substitutions at position 205 from a cysteine to a tyrosine C205Y, and at position 231 from a tyrosine to an Asparagine Y231N, and
wherein the lettuce plant further comprises the resistance gene LG7 that encodes for the resistance protein LG7 having at least 99% sequence identity with SEQ ID NO: 8, and wherein said resistance protein LG7 has a valine (V) at position 111, and wherein the lettuce plant is selected from the group consisting of Lactuca sativa, Lactuca virosa, Lactuca saligna, Lactuca serriola, Lactuca aculeate, Lactuca georgica, Lactuca perennis, Lactuca tatarica, and Lactuca viminea.

24. The method according to claim 23, wherein the one or more mutations in the KINASE gene results in a protein set forth in SEQ ID NO: 4.

25. (canceled)

26. The method according to claims 23, wherein the one or more mutations in the KINASE gene are obtained by gene editing techniques.

27. A method for identifying or selecting (i) a Fusarium wilt resistant lettuce plant or (ii) a seed of said plant according to claim 1, wherein the method comprises the step of establishing, in a genome of said plant or seed, the presence of a resistance gene KINASE R, or a resistance gene LG7, or a resistance locus LG7, or a combination of a resistance gene KINASE R with a resistance locus LG7 or the resistance gene LG7 in a lettuce plant,
wherein:
(a) the resistance gene LG7 confers resistance to Fusarium wilt in lettuce plants, wherein the resistance gene LG7 comprises a coding sequence that has at least 99% sequence identity with SEQ ID NO: 7, or wherein the resistance gene LG7 encodes for a resistance protein LG7 having at least 99% sequence identity with SEQ ID NO: 8; or
(b) the combination of the resistance gene KINASE R with the resistance locus LG7 or the resistance gene LG7 in the lettuce plant confers resistance to Fusarium wilt to said lettuce plant, wherein:
the resistance gene KINASE R comprises a coding sequence having at least 99% sequence identity with SEQ ID NO: 3, and wherein said resistance locus LG7 comprises said resistance gene LG7, wherein said resistance gene LG7 comprises a coding sequence having at least 99% sequence identity with SEQ ID NO: 7; or
the resistance gene KINASE R encodes for a KINASE protein sequence having at least 99% sequence identity with SEQ ID NO: 4, and wherein said resistance locus LG7 comprises said resistance gene LG7, wherein said resistance gene LG7 encodes for a resistance protein LG7 having at least 99% sequence identity with SEQ ID NO: 8.

28. A method, comprising using the combination according to claim 17 for providing Fusarium resistance in lettuce plants.

29. A method, comprising using a plasmid for introducing a combination of a resistance gene KINASE R with a resistance gene LG7 or a resistance locus LG7 into the genome of a lettuce plant or lettuce plant cell, wherein the plasmid comprises the combination according to claim 17.


SEQUENCE LISTING (8 sequences)

SEQ ID NO: 1 - DNA, 2469 bp, Lactuca sativa
  misc_feature 1..2469: note = Kinase
  source 1..2469: mol_type = other DNA
atgaacttttctacagctcttatatttctccttactgtattggatattcttgtttattatttacagaaacaacacgaggaagacaacggtgagcagaagaaatcatccctgtcatcatcatcatcggatcaacaactatcgctacacgaagaagaacatgatcagaggaaggatttatcttcatcacggctactggataaaagatgtcgatgtttttcacttgctgaaatcaaattggccaccaatgattttgatgatgcgtttgtaatcggaaagggagggtttgggaaggtatacaaaggtaagattgacttcggggaagaaggaattgatgttgctattaagaggttgaatcttgactccaatcaaggagtaaccgagttcaaggcagagattgagatgctttccaagtttcgccatagtcacatcgtctctcttttaggataccatgaaggatccgataaaagagaaatgatcattgtttatgaatacatgcctaatggaagtcttgaggatcatttgcataaaagaaaagttaacggaagtaattcctctcttctgacgtgggtccaaacgctccaaatctgcataggtgctgctcgtggtttggactgccttcattcgggtacaggtgtcagatccagagtcatacaccgtgatatcaagagctcaaacatcttgttggacgagtatttagcagctaaagtttctgacttcgggttgtccaggattggtccagcaaatctggtggggaccactaatgtttatactgaccaaatcaaaggaacatttgggtacatggatgctgagtatttttcaacttgtagactgacacgaaagtctgatgtgtatgcatttggtgtggtgttattggaagtgttatgtggtaggcctgctctggattttacattagatgagcagcaacatagtctagctgtatgggctaaaagctgcatcaaagaaggtaagattgatcaaatcattgatcaatgtttaaagggacaaacaacaggtaattgtgttaaagaatttgggaggattgcatatgaatgtgttcttactcgttcaaaggatcgacccacaatgagtcaggtggtagctaggcttgagtttgtgttagcatggacactgcaaagtgggcccagtgctcgtgatcgaaaacaaattggacgagccatgtttattgaaaaagcatggtcattgttcttgattaaatctccaaaaagtcgtatgagttctcctacaaagaaactaggacaaagtatttacaacaaaaagacaagtgttgcaactggttctcagaaggtgttgctacctaatgctaacacaaagataccaatcttgaagatgtttacattttctgagctacaaagtgcaacaaagaatttcaagcaggagatgtttcttggagaaggtggttatgggaaggtttacaaagggtggttggacagtgtaacatttgccccacgaaagccaggtgatgggcttgctgtggccattaagagatccagccccaacaggactcaaggtcttaatgaatggcaggcagaagtgaaattcttgggaacatttagccacccaaacattgttaaactctttggatattgctgggaaaacaaggagtttcttcttgtttatgaatttatgcaaaaaggaagtttagatatgcaactcttcagagaaggtgtagaaccacttccatgggacacaaggattaaaatagccacaggagcagctcaaggccttgctttcttgcacactacagaaaataatgtcatttgtagggatgtgaagtcctcaaatattttactagatggggagttcaatgcaaagctttccgattttggacttgcaaagtcaggtcctgtcaaaggagagtctcatgtctcaacagacatagcaggcacctatggttacatggctcctgagtatattgccactggtcgtttatatgtgaagagtgatgtttatggttttggggtgttgatgttggagatcataacagggttacgcgcatctgattataatcaagatggcatgaagcaaaacttggtggaatgggctacaccttttttaactgatataaagaaattggaaaaaattatggatccacgtttgggacaaaactacccttcagaaggtgctagtaaagcagctgagctcatactaagttgccttgattcggaccctaaaaaccggccttccatggaggaagttgtattgggtttgcaagttattggttcggtcaaaatggaaccagggcggtcaacgtctagcaccaggggttcgatgagcccatgcagcgaacaaagttgcccttggtctccccttcgtggaaagcaagggagggcagtttggtcaatgagcactcggttgttaggtttaaatgtgtgccttggactcggttcaaaggaagacagaacttggatggaaacaaactgttccaaaaccagaaaatggggaaattga

SEQ ID NO: 2 - protein, 822 aa, Lactuca sativa
  REGION 1..822: note = Kinase protein
MNFSTALIFLLTVLDILVYYLQKQHEEDNGEQKKSSLSSSSSDQQLSLHEEEHDQRKDLSSSRLLDKRCRCFSLAEIKLATNDFDDAFVIGKGGFGKVYKGKIDFGEEGIDVAIKRLNLDSNQGVTEFKAEIEMLSKFRHSHIVSLLGYHEGSDKREMIIVYEYMPNGSLEDHLHKRKVNGSNSSLLTWVQTLQICIGAARGLDCLHSGTGVRSRVIHRDIKSSNILLDEYLAAKVSDFGLSRIGPANLVGTTNVYTDQIKGTFGYMDAEYFSTCRLTRKSDVYAFGVVLLEVLCGRPALDFTLDEQQHSLAVWAKSCIKEGKIDQIIDQCLKGQTTGNCVKEFGRIAYECVLTRSKDRPTMSQVVARLEFVLAWTLQSGPSARDRKQIGRAMFIEKAWSLFLIKSPKSRMSSPTKKLGQSIYNKKTSVATGSQKVLLPNANTKIPILKMFTFSELQSATKNFKQEMFLGEGGYGKVYKGWLDSVTFAPRKPGDGLAVAIKRSSPNRTQGLNEWQAEVKFLGTFSHPNIVKLFGYCWENKEFLLVYEFMQKGSLDMQLFREGVEPLPWDTRIKIATGAAQGLAFLHTTENNVICRDVKSSNILLDGEFNAKLSDFGLAKSGPVKGESHVSTDIAGTYGYMAPEYIATGRLYVKSDVYGFGVLMLEIITGLRASDYNQDGMKQNLVEWATPFLTDIKKLEKIMDPRLGQNYPSEGASKAAELILSCLDSDPKNRPSMEEVVLGLQVIGSVKMEPGRSTSSTRGSMSPCSEQSCPWSPLRGKQGRAVWSMSTRLLGLNVCLGLGSKEDRTWMETNCSKTRKWGN

SEQ ID NO: 3 - DNA, 2469 bp, Lactuca sativa
  misc_feature 1..2469: note = Kinase R
  source 1..2469: mol_type = other DNA
atgaacttttctacagctcttatatttctccttactgtattggatattcttgtttattatttacagaaacaacacgaggaagacaacggtgagcagaagaaatcatccctgtcatcatcatcatcggatcaacaactatcgctacacgaagaagaacatgatcagaggaaggatttatcttcatcacggctactggataaaagatgtcgatgtttttcacttgctgaaatcaaattggccaccaatgattttgatgatgcgtttgtaatcggaaagggagggtttgggaaggtatacaaaggtaagattgacttcggggaagaaggaattgatgttgctattaagaggttgaatcttgactccaatcaaggagtaaccgagttcaaggcagagattgagatgctttccaagtttcgccatagtcacatcgtctctcttttaggataccatgaaggatccgataaaagagaaatgatcattgtttatgaatacatgcctaatggaagtcttgaggatcatttgcataaaagaaaagttaacggaagtaattcctctcttctgacgtgggtccaaacgctccaaatctgcataggtgctgctcgtggtttggactaccttcattcgggtacaggtgtcagatccagagtcatacaccgtgatatcaagagctcaaacatcttgttggacgagaatttagcagctaaagtttctgacttcggcttgtccaggattggtccagcaaatctggtggggaccactaatgtttatactgaccaaatcaaaggaacatttgggtacatggatgctgagtatttttcaacttgtagactgacacgaaagtctgatgtgtatgcatttggtgtggtgttattggaagtgttatgtggtaggcctgctctggattttacattagatgagcagcaacatagtctagctgtatgggctaaaagctgcatcaaagaaggtaagattgatcaaatcattgatcaatgtttaaagggacaaacaacaggtaattgtgttaaagaatttgggaggattgcatatgaatgtgttcttactcgttcaaaggatcgacccacaatgagtcaggtggtagctaggcttgagtttgtgttagcatggacactgcaaagtgggcccagtgctcgtgatcgaaaacaaattggacgagccatgtttattgaaaaagcatggtcattgttcttgattaaatctccaaaaagtcgtatgagttctcctacaaagaaactaggacaaagtatttacaacaaaaagacaagtgttgcaactggttctcagaaggtgttgctacctaatgctaacacaaagataccaatcttgaagatgtttacattttctgagctacaaagtgcaacaaagaatttcaagcaggagatgtttcttggagaaggtggttatgggaaggtttacaaagggtggttggacagtgtaacatttgccccacgaaagccaggtgatgggcttgctgtggccattaagagatccagccccaacaggactcaaggtcttaatgaatggcaggcagaagtgaaattcttgggaacatttagccacccaaacattgttaaactctttggatattgctgggaaaacaaggagtttcttcttgtttatgaatttatgcaaaaaggaagtttagatatgcaactcttcagagaaggtgtagaaccacttccatgggacacaaggattaaaatagccacaggagcagctcaaggccttgctttcttgcacactacagaaaataatgtcatttgtagggatgtgaagtcctcaaatattttactagatggggagttcaatgcaaagctttccgattttggacttgcaaagtcaggtcctgtcaaaggagagtctcatgtctcaacagacatagcaggcacctatggttacatggctcctgagtatattgccactggtcgtttatatgtgaagagtgatgtttatggttttggggtgttgatgttggagatcataacagggttacgcgcatctgattataatcaagatggcatgaagcaaaacttggtggaatgggctacaccttttttaactgatataaagaaattggaaaaaattatggatccacgtttgggacaaaactacccttcagaaggtgctagtaaagcagctgagctcatactaagttgccttgattcggaccctaaaaaccggccttccatggaggaagttgtattgggtttgcaagttattggttcggtcaaaatggaaccagggcggtcaacgtctagcaccaggggttcgatgagcccatgcagcgaacaaagttgcccttggtctccccttcgtggaaagcaagggagggcagtttggtcaatgagcactcggttgttaggtttaaatgtgtgccttggactcggttcaaaggaagacagaacttggatggaaacaaactgttccaaaaccagaaaatggggaaattga

SEQ ID NO: 4 - protein, 822 aa, Lactuca sativa
  REGION 1..822: note = Kinase R protein
MNFSTALIFLLTVLDILVYYLQKQHEEDNGEQKKSSLSSSSSDQQLSLHEEEHDQRKDLSSSRLLDKRCRCFSLAEIKLATNDFDDAFVIGKGGFGKVYKGKIDFGEEGIDVAIKRLNLDSNQGVTEFKAEIEMLSKFRHSHIVSLLGYHEGSDKREMIIVYEYMPNGSLEDHLHKRKVNGSNSSLLTWVQTLQICIGAARGLDYLHSGTGVRSRVIHRDIKSSNILLDENLAAKVSDFGLSRIGPANLVGTTNVYTDQIKGTFGYMDAEYFSTCRLTRKSDVYAFGVVLLEVLCGRPALDFTLDEQQHSLAVWAKSCIKEGKIDQIIDQCLKGQTTGNCVKEFGRIAYECVLTRSKDRPTMSQVVARLEFVLAWTLQSGPSARDRKQIGRAMFIEKAWSLFLIKSPKSRMSSPTKKLGQSIYNKKTSVATGSQKVLLPNANTKIPILKMFTFSELQSATKNFKQEMFLGEGGYGKVYKGWLDSVTFAPRKPGDGLAVAIKRSSPNRTQGLNEWQAEVKFLGTFSHPNIVKLFGYCWENKEFLLVYEFMQKGSLDMQLFREGVEPLPWDTRIKIATGAAQGLAFLHTTENNVICRDVKSSNILLDGEFNAKLSDFGLAKSGPVKGESHVSTDIAGTYGYMAPEYIATGRLYVKSDVYGFGVLMLEIITGLRASDYNQDGMKQNLVEWATPFLTDIKKLEKIMDPRLGQNYPSEGASKAAELILSCLDSDPKNRPSMEEVVLGLQVIGSVKMEPGRSTSSTRGSMSPCSEQSCPWSPLRGKQGRAVWSMSTRLLGLNVCLGLGSKEDRTWMETNCSKTRKWGN

SEQ ID NO: 5 - DNA, 121 bp, synthetic construct
  misc_feature 1..121: note = Marker 1 LG7
  source 1..121: mol_type = other DNA
agggaaggacttaaggtcctttaaatatggtgcaaaacctaaaaattagggttttcatgttcagcctcaactcgtcgagtaggttctagaatccatgcggttttatcagtttgtacacgac

SEQ ID NO: 6 - DNA, 121 bp, synthetic construct
  misc_feature 1..121: note = Marker 2 LG7
  source 1..121: mol_type = other DNA
ccaggaagtcaaatgaactcccaggtgtggctcagatagccaaaaaaatttgataggttcataccctcaagtatgtggggtttatggtcgtagcataaattctgctagacaaggaattcag

SEQ ID NO: 7 - DNA, 747 bp, Lactuca sativa
  source 1..747: mol_type = other DNA
atggaaggtttcaaggatctcaccatacagttagcacaatattgtgctggaaacccactagctcttaaagtattgggttcttcttcattttttgatgttgaagagccaaaggaaaaaaataacatgatagaggtttggagaagtaaattggattcactgaattcattaaaaggagatcttcattgtaaaatccaaggtatacttcaaaagagctttgactcgttaccacatgccagtaacaaagagttgtttctgcatactacttttttctttgtcggtgaatatgagggttatgtggtaaagatattggagcgtgactggaatgcaaaggttaggatcaggaacctcattaatagatgccttcttaccatctcaccaaggaaaaaactgatgatgtatcaactgcttcaagagatggaaagaaacatagtccttcaagagtccagcgatcccgcagcacgtagtagagtctcacaaaatgatgaatcttatcgtttgttgacaaaaggagagccaacaacccttaaaacaacttcacttgcaaaaacggacaaactaaaatttctcaagataaaacatgtggaactcaaggggtcctacaagaattttccagaattaagatggttgtactggagtgagtatcaaatgaaaagaataccctccggcttatgggggggcaacatggtggctatagatatgagcaatggatggttgaagaagttcgaaccacccatggtaggaatttga

SEQ ID NO: 8 - protein, 248 aa, Lactuca sativa
MEGFKDLTIQLAQYCAGNPLALKVLGSSSFFDVEEPKEKNNMIEVWRSKLDSLNSLKGDLHCKIQGILQKSFDSLPHASNKELFLHTTFFFVGEYEGYVVKILERDWNAKVRIRNLINRCLLTISPRKKLMMYQLLQEMERNIVLQESSDPAARSRVSQNDESYRLLTKGEPTTLKTTSLAKTDKLKFLKIKHVELKGSYKNFPELRWLYWSEYQMKRIPSGLWGGNMVAIDMSNGWLKKFEPPMVGI